(19) United States
(12) Patent Application Publication
(10) Pub. No.: US 2012/0220855 A1
(43) Pub. Date: Aug. 30, 2012

(54) Method and System for MR Scan Range Planning

(75) Inventors: Wei Zhang, Falls Church, VA (US); Michael Suehling, Erlangen (DE); Shaohua Kevin Zhou, Plainsboro, NJ (US)

(73) Assignee: Siemens Corporation, Iselin, NJ (US)

(21) Appl. No.: 13/033,976

(22) Filed: Feb. 24, 2011

(51) Int. Cl. A61B 5/055

(52) U.S. Cl. 600/410

(57) ABSTRACT

A method and system for determining a scan range for a magnetic resonance (MR) scan is disclosed. A plurality of 2D localizer images are received. A most likely position is detected in each localizer image for each of a plurality of anatomical landmarks associated with a target organ in each localizer image. A scan range is determined based on the detected most likely positions of each anatomic landmark in the localizer images.

### BACKGROUND OF THE INVENTION

The present invention relates to medical imaging of a patient, and more particularly, to automatically defining a scan range for a magnetic resonance (MR) scan of the liver based on two-dimensional (2D) localizer images.

Magnetic Resonance (MR) is a well known technique for imaging internal organs of a patient. When targeting a specific organ, such as the liver, with an MR scan, a scan range must be determined for the MR scan. The scan range determines where, relative to the patient's body, the MR scan begins and ends. If the scan range is too small, a portion of the organ may be missed, which can lead to the loss of important information. If the scan range is too large, the MR scan will acquire extra information that is not necessary. Since typical high definition MR scans are relatively slow, scanning a larger range than is necessary is inefficient. In addition to additional patient discomfort caused by long scanning times, the MR scanner is unnecessarily occupied leading to a lower utilization capacity.

In conventional MR scans, the scan range is typically determined manually by experienced MR operators. For example, in conventional MR scanning, scout/localizer images may be obtained using lower resolution scans that are acquired first to let MR operators plan the subsequent diagnostic scans. The diagnostic scans typically have a higher resolution and better contrast and are obtained by sequences requiring much longer time. In order to determine a scan range for a diagnostic scan, a MR operator typically manually determines a range that includes the targeted organ by looking at a localizer image. However, this process may be inaccurate, time consuming, and inconsistent.

### BRIEF SUMMARY OF THE INVENTION

The present invention provides a method and system for automatic magnetic resonance (MR) scan range planning. Embodiments of the present invention automatically detect a scan range for an MR liver scan based on 2D localizer images. Embodiments of the present invention automatically detect anatomical structures in 2D localizer images and determine the scan range based on the detected anatomical structures. Embodiments of the present invention can be applied to MR data acquired with different protocols, such as different echo time, repetition time, magnetic strength, etc.

In one embodiment of the present invention, most likely positions of anatomic landmarks are detected in each of a plurality of 2D localizer images. The most likely positions of the anatomic landmarks can be detected in each localizer image using learning based landmark detectors and a discriminative anatomical network. A scan range is determined based on the detected most likely positions of the landmarks in each of the plurality of 2D localizer images. The scan range can be determined by removing outliers from the detected most likely landmark positions and selecting the detected most likely position for each landmark to define a scan range that encompasses all remaining detected most likely potions of the landmarks.

These and other advantages of the invention will be apparent to those of ordinary skill in the art by reference to the following detailed description and the accompanying drawings.

### BRIEF DESCRIPTION OF THE DRAWINGS

FIG. 1 illustrates a method of automatically determining a scan range for an MR scan according to an embodiment of the present invention;

FIG. 2 illustrates exemplary 2D localizer images;

FIG. 3 illustrates a method of detecting anatomic landmarks in a localizer image according to an embodiment of the present invention;

FIG. 4 illustrates a method of determining a scan range based on detected landmarks in localizer images according to an embodiment of the present invention;

FIGS. 5 and 6 illustrate exemplary scan range determination results; and

FIG. 7 is a high level block diagram of a computer capable of implementing the present invention.

### DETAILED DESCRIPTION

The present invention is directed to a method and system for automatic determination of a scan range for a magnetic resonance (MR) scan of a targeted organ using 2D localizer MR images. An advantageous embodiment of the present invention automatically determines a scan range for an MR liver scan. Embodiments of the present invention are described herein to give a visual understanding of the scan range determination method. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, it is to be understood that embodiments of the present invention may be performed within a computer system using data stored within the computer system.

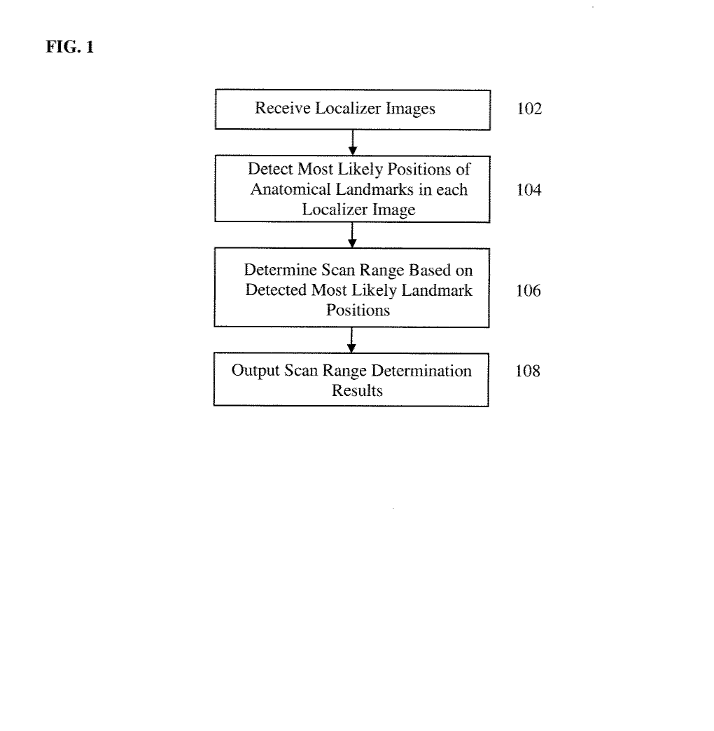

FIG. 1 illustrates a method of automatically determining a scan range for an MR scan according to an embodiment of the present invention. The method of FIG. 1 transforms medical image data representing anatomy of a patient to detect a particular set of anatomic landmarks in the medical image data and to determine a scan range for an MR scan based on the detected anatomic landmarks. The method of FIG. 1 detects particular anatomic landmarks that define a scan range for a particular organ. For example, in liver scan range detection, the liver dome and right lobe lower tip of the liver are detected and used to define a scan range.

At step 102, a plurality of localizer images are received. The localizer images are 2D MR images obtained using lower resolution scans that are much quicker than a high resolution diagnostic scans. The localizer images can be received directly from an MR scanner. It is also possible that the localizer images can be received by loading localizer images that were previously stored, for example on a memory or storage of a computer system or a computer readable medium. According to one embodiment the plurality of localizer images can include each 2D slice of an MR scan. Since it is not known a priori which slices contain the anatomical structures that are used to define the scan range, each slice in an MR scan may be scanned to determine the best positions of the anatomical structures. Further, the plurality of slices may include slices obtained with different orientations, such as coronal, sagittal, and axial slices. For example, in liver scan range detection, the plurality of slices includes at least a plurality of coronal slices and a plurality of sagittal slices. Spatial correspondence between the different slices is given by the DICOM Patient Coordinate System contained in the DICOM header information of each of the slices.

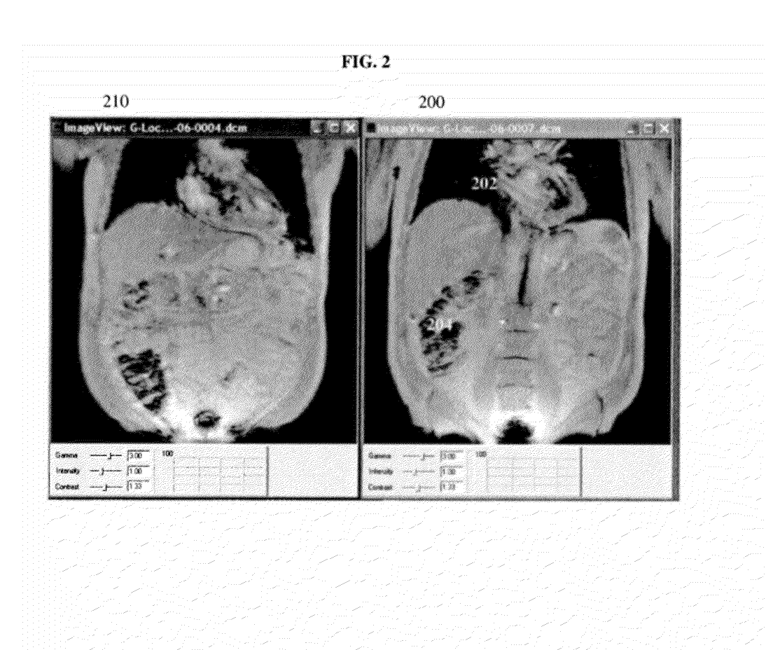

FIG. 2 illustrates exemplary 2D localizer images. As illustrated in FIG. 2, localizer images 200 and 210 are coronal slices of an MR scan. The positions of the liver dome 202 and the right lobe lower tip 204 of the liver are shown in image 200. The liver dome 202 and right lobe tip 204 may exist in only one coronal slice. For example, in the liver dome and right lobe tip cannot be seen in image 210.

Returning to FIG. 1, at step 104, most likely positions are detected in each of the plurality of localizer images for anatomical landmarks associated with a target organ. According to an advantageous embodiment in which the target organ is the liver, the most likely position of the liver dome and right lobe lower tip of the liver are detected in each localizer image. The most likely position for each anatomical landmark (structure) can be detected in an image using learning based detectors. A separate landmark detector is trained for each anatomic landmark based on annotated training data.

According to an advantageous implementation, each landmark detector can be trained using a probabilistic boosting cascade tree (PBCT) framework. Training a detector using the PBCT framework is described in detail in United States Published Patent Application No. 2008/0071711, which is incorporated herein by reference. Each trained landmark detector may utilize a marginal space learning (MSL) detection scheme. In order to detect a structure, MSL decomposes the parameter space of the structure along decreasing levels of geometrical abstraction into subspaces of increasing dimensionality by exploiting parameter invariance. At each level of abstraction, i.e., in each subspace, strong discriminative models are trained from annotated training data (e.g., using a probabilistic boosting tree (PBT) or PBCT), and these models are used to narrow the range of possible solutions until a final position of the structure can be inferred. The basic MSL framework is described in greater detail in United States Published Patent Application No. 2008/0101676, which is incorporated herein by reference. When training each landmark detector positive training samples are generated based on human annotations of training images. Negative training samples are generated randomly from the background area of the annotated training images. In order to suppress false positives from slices which do not contain the particular structure, negative training samples are also collected from such irrelevant slices. As described above, the plurality of localizer images may include localizer images obtained using various orientations (e.g., coronal, sagittal, and axial slices). Separate landmarks detectors for each landmark can be trained for each orientation. For example, in liver scan range detection, separate liver dome detectors can be trained for coronal and sagittal slices and separate right lobe lower tip detectors can be trained for coronal and sagittal slices.

According to an advantageous implementation, rather than treat individual structure detection independently, the anatomical structures can be detected sequentially, ordered by their detection reliability. The detection reliability depends on each structure's appearance variation. For example, the right lobe lower tip area of the liver is much more complicated than the liver area. Consequently, the reliability of the liver dome detector is higher. Accordingly, the liver dome can be detected first and the search range for detection of the right lobe lower tip can be constrained based on the detected liver dome position. Each anatomic landmark detector can return a certain number of position candidates for each localizer image. For example each landmark detector can return up to ten candidates for the most likely position of the corresponding landmark in a localizer image. A discriminative anatomical network (DAN) can then be used to consider the joint relationship between the anatomical landmarks in order to select the best candidates for each landmark in each localizer image.

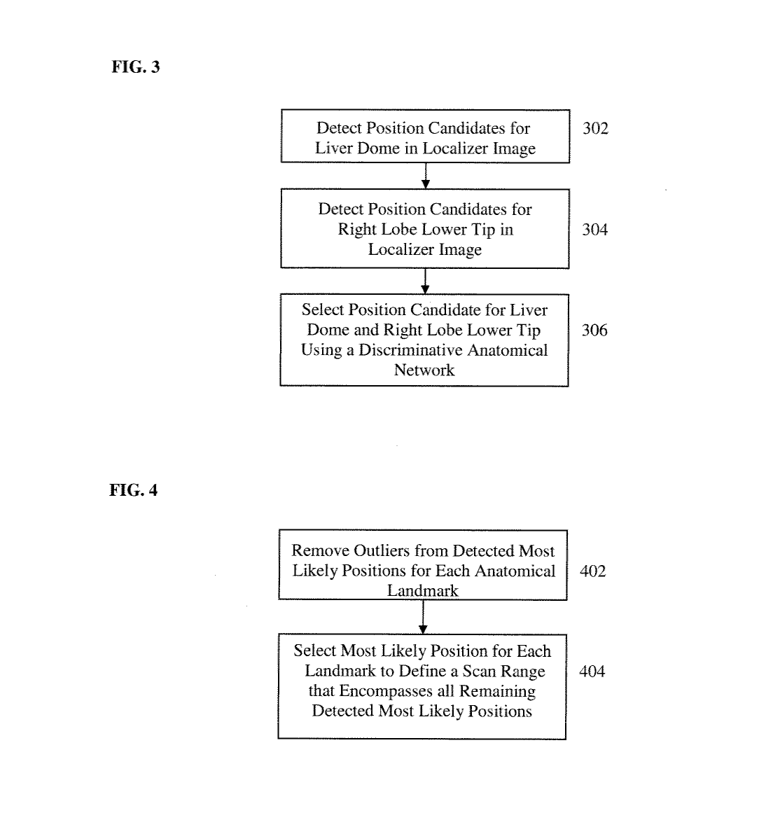

FIG. 3 illustrates a method of detecting anatomical landmarks associated with the liver in a 2D localizer image. The method of FIG. 3 can be used for each of the received localizer images in order to implement step 104 of the method of FIG. 1. Referring to FIG. 3, at step 302, position candidates of the liver dome are detected in the localizer image using a trained liver dome detector. As described above, the liver dome detector can de trained based on annotated training data using a PBCT. The liver dome detector can scan the localizer image and return up to a certain number of position candidates for the liver dome. For example, the liver dome detector may return up to ten best position candidates for the liver dome.

At step 304, position candidates of the right lobe lower tip of the liver are detected in the localizer image using a trained right lobe lower tip detector constrained based on the detected position candidates of the liver dome. The search range for the right lobe lower tip detector in the localizer image can be determined from the position candidates of the liver dome based on prior information collected using the annotated training data. In particular, a search range can de determined from the detected position candidates of the liver dome based on the relative distance between the liver dome and the right lobe lower tip and the standard deviations in the annotated training data. As described above, the right lobe lower tip detector can be trained using a PBCT. The right lobe lower tip dome detector can scan the constrained search range of the localizer image and return up to a certain number of position candidates for the right lobe lower tip. For example, the right lobe lower tip detector may return up to ten best position candidates for the right lobe lower tip.

At step 306, one of the position candidates is selected for each of the liver dome and the right lobe lower tip using a DAN. The DAN considers the joint relationship between the liver dome and the right lobe lower tip in order find the best landmark configuration. In order to reduce the network complexity for a possible large number of structures, the whole network can be divided into one or more sub-networks. Within a sub-network, the optimal solution is searched exhaustively. The DAN is based on pairwise potentials defined based on the vector between landmarks. For each sub-network, the optimal solution is the one which maximizes the following distribution:

$$p ( x ) = {\prod }_{u ∈ S}^{ } {ϕ}_{u} ( {l}_{u} ) {\prod }_{u ∈ S , v ∈ S}^{ } {ϕ}_{uv} ( {l}_{u} , {l}_{v} ) {\prod }_{u ∈ S , v ∈ d ( S )}^{ } {ρ}_{uv} ( {l}_{u} , {l}_{v} )$$

where S represents the set of landmarks belonging to the sub-net, d(S) represents a sub-network on which the current sub-network depends, and ρᵤᵥ(lᵤ,lᵥ) is a pairwise potential across two different sub-networks, for which a combinatory search is not needed. Once the previous sub-network is optimized, the configuration within that sub-network is fixed. It is to be understood that when only two structures are detected, as in the method of FIG. 3, there is only one sub-network.

Although described herein with respect to only two landmarks, an advantage of the DAN is that it can easily scale up to additional landmarks without exponentially increasing in complexity, while still jointly considering landmarks which are locally coupled, as well as the belief propagated from more stable landmarks.

Returning to FIG. 1, at step 106, a scan range is determined based on the most likely landmark positions detected in each of the plurality of localizer images. In order to determine the best scan range based on the detected most likely landmark positions, the landmark detections from each localizer image are put onto a common coordinate system and one of the detected landmark positions is selected for each landmark. The scan range is defined based on the selected landmark positions for each landmark.

FIG. 4 illustrates a method for determining a scan range based on a plurality of detected most likely landmark positions. The method of FIG. 4 can be used to implement step 106 of the method of FIG. 1. Referring to FIG. 4, at step 402, outliers are removed from the detected most likely positions for each landmark. In order to remove outliers for a particular landmark, the median position of the detected structures in all of the localizer images is determined and detections that are greater than a certain distance from the median position are removed. For example, in liver scan range detection, the median position of the detected most likely liver dome positions can be determined and any detected most likely liver dome positions that are far away from the median position are removed. Similarly, the median position of the detected most likely right lobe lower tip positions can be determined and any detected most likely right lobe lower tip positions that are far away from the median position are removed.

At step 404, one of the most likely positions is selected for each landmark to define a scan range that encompasses all of the remaining detections. The outermost most likely position is selected for each landmark so that a scan range that is defined by the selected landmark positions encompasses all remaining most likely position detections. This ensures that the entire target organ is included in the scan range. For example, in the liver scan range determination, an uppermost most likely position is selected for the liver dome and a lowermost most likely position is selected for the right lobe lower tip. The scan range is defined as between the uppermost detected liver dome and the lowermost detected right lobe lower tip.

Returning to FIG. 1, at step 108, the scan range determination results are output. For example the positions of the landmarks determined in step 106, which defined the scan range can be output. The scan range determination results can be output by displaying the positions of the landmarks and the determined scan range, for example, on a display of a computer. The scan range determination results can be output by storing the results on a memory or storage of a computer device or on a computer readable storage medium. The determined scan range can be output to an MR scanner in order to set the range of a high resolution diagnostic MR scan. The MR scanner can them perform the diagnostic scan using the determined scan range.

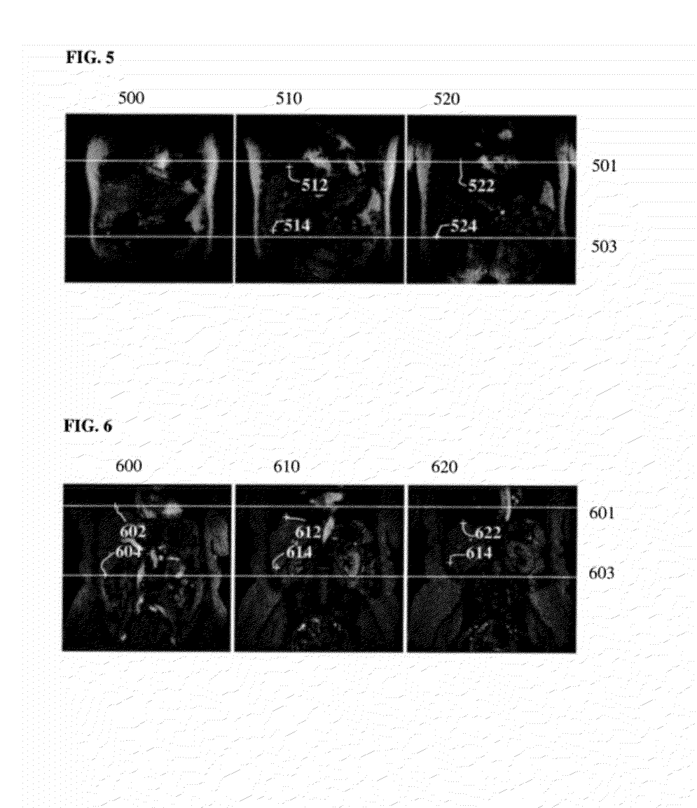

FIGS. 5 and 6 show exemplary scan range determination results using MR images acquired with different protocols. FIG. 5 illustrates 2D MR slices 500, 510, and 520 acquired with an echo time of 5.0 ms, a repetition time of 15.0 ms, a magnetic strength of 1.494000 T, a slice thickness of 10.0 mm, and a spacing between slices of 15.0 mm. Crosses 512 and 514 indicate the detected most likely positions of the liver dome and right lobe lower tip, respectively, in slice 510. Crosses 522 and 524 indicate the detected most likely positions of the liver dome and right lobe lower tip, respectively, in slice 520. No most likely positions were detected for the liver dome and the right lobe lower tip in slice 500. Lines 501 and 503 show the final determined scan range. The liver dome position 522 detected in slice 520 is the uppermost liver dome position and the right lobe lower tip position 524 detected in slice 520 is the lowermost right lobe lower tip position. Accordingly, the liver dome position 522 and the right lobe lower tip position 524 in slice 520 define the search range.

FIG. 6 illustrates 2D MR slices 600, 610, and 620 acquired with an echo time of 3.69 ms, a repetition time of 7.8 ms, a magnetic strength of 3.0 T, a slice thickness of 6.0 mm, and a spacing between slices of 7.8 mm. Crosses 602 and 604 indicate the detected most likely positions of the liver dome and right lobe lower tip, respectively, in slice 6000. Crosses 612 and 614 indicate the detected most likely positions of the liver dome and right lobe lower tip, respectively, in slice 610. Crosses 622 and 624 indicate the detected most likely positions of the liver dome and right lobe lower tip, respectively, in slice 620. Lines 601 and 603 show the final determined scan range. As shown in FIG. 5, the liver dome position 602 detected in slice 600 is the uppermost liver dome position and the right lobe lower tip position 6004 detected in slice 6000 is the lowermost right lobe lower tip position. Accordingly, the liver dome position 602 and right lobe lower tip position 604 on slice 600 define the search range.

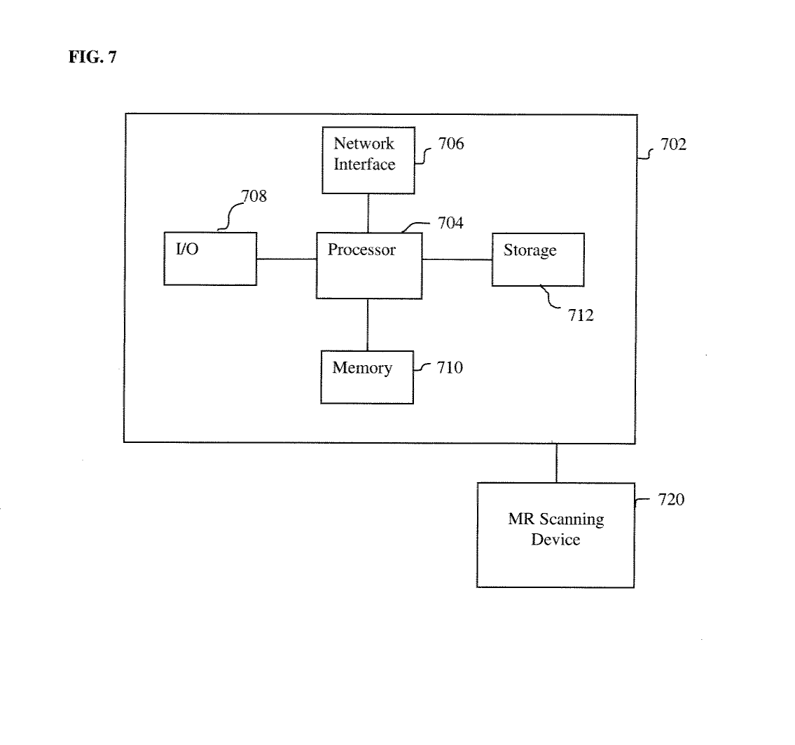

The above-described methods for determining a scan range for an MR scan of a target organ may be implemented on a computer using well-known computer processors, memory units, storage devices, computer software, and other components. A high level block diagram of such a computer is illustrated in FIG. 7. Computer 702 contains a processor 704 which controls the overall operation of the computer 702 by executing computer program instructions which define such operations. The computer program instructions may be stored in a storage device 712, or other computer readable medium (e.g., magnetic disk, CD ROM, etc.) and loaded into memory 710 when execution of the computer program instructions is desired. Thus, the steps of the methods of FIGS. 1, 3, and 4 may be defined by the computer program instructions stored in the memory 710 and/or storage 712 and controlled by the processor 704 executing the computer program instructions. An MR scanning device 720 can be connected to the computer 702 to input medical images to the computer 702. It is possible to implement the MR scanning device 720 and the computer 702 as one device. It is also possible that the MR scanning device 720 and the computer 702 communicate wirelessly through a network. The computer 702 also includes one or more network interfaces 706 for communicating with other devices via a network. The computer 702 also includes other input/output devices 708 that enable user interaction with the computer 702 (e.g., display, keyboard, mouse, speakers, buttons, etc.). One skilled in the art will recognize that an implementation of an actual computer could contain other components as well, and that FIG. 7 is a high level representation of some of the components of such a computer for illustrative purposes.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

1. A method for detecting a scan range for a magnetic resonance (MR) scan of a target organ based on a plurality of 2D MR localizer images, comprising:
detecting a most likely position of each of a plurality of anatomical landmarks associated with a target organ in each of the plurality of 2D MR localizer images; and
determining a scan range for the target based on the detected most likely positions of each of the plurality of anatomical landmarks in the plurality of 2D MR localizer images.

2. The method of claim 1, wherein the step of detecting a most likely position of each of a plurality of anatomical landmarks associated with a target organ in each of the plurality of 2D MR localizer images comprises:
detecting one or more most likely position candidates for each anatomical landmark in each of the plurality of 2D MR localizer images using a separate trained landmark detector for each anatomic landmark.

3. The method of claim 2, wherein the step of detecting one or more most likely position candidates for each anatomical landmark in each of the plurality of 2D MR localizer images using a separate trained landmark detector for each anatomic landmark comprises:
detecting the most likely position candidates for each anatomical landmark sequentially based on a detection reliability of each trained landmark detector, wherein a search range for detecting at least one subsequent anatomical landmark is constrained based on the detected position candidates for at least one previous anatomical landmark.

4. The method of claim 2, wherein the step of detecting a most likely position of each of a plurality of anatomical landmarks associated with a target organ in each of the plurality of 2D MR localizer images further comprises:
selecting one of the most likely position candidates for each anatomical landmark based on a relationship between the anatomical landmarks using a discriminative anatomical network.

5. The method of claim 1, wherein the step of determining a scan range for the target based on the detected most likely positions of each of the plurality of anatomical landmarks in the plurality of 2D MR localizer images comprises:
removing outliers from the detected most likely positions for each of the plurality of anatomical landmarks; and
selecting one of the most likely positions for each of the anatomical landmarks to define a search range, such that the search range defined by the selected most likely position of each of the anatomical landmarks encompasses all of the other detected most likely positions for each anatomical landmark.

6. The method of claim 5, wherein the step of removing outliers from the detected most likely positions for each of the plurality of anatomical landmarks comprises:
determining a median position of the detected most likely positions for each anatomical landmark; and
removing detected most likely positions of each anatomical landmark that are more than a certain distance from the median position determined for that anatomical landmark.

7. The method of claim 5, wherein the step of selecting one of the most likely positions for each of the anatomical landmarks to define a search range comprises:
selecting an outermost most likely position for each of the anatomical landmarks.

8. The method of claim 1, wherein the target organ is a liver and the plurality of anatomical landmarks comprises a liver dome and a right lobe lower tip.

9. The method of claim 8, wherein the step of detecting a most likely position of each of a plurality of anatomical landmarks associated with a target organ in each of the plurality of 2D MR localizer images comprises:
detecting one or more most likely position candidates for the liver dome in each of the plurality of 2D MR localizer images using a trained liver dome detector;
detecting one or more most likely position candidates for the right lobe lower tip in each of the plurality of 2D MR localizer images using a trained right lobe lower tip detector constrained based on the detected most likely position candidates for the liver dome; and
selecting one of the most likely position candidates for the liver dome and one of the most likely position candidates for the right lobe lower tip using a discriminative anatomical network.

10. The method of claim 8, wherein the step of determining a scan range for the target based on the detected most likely positions of each of the plurality of anatomical landmarks in the plurality of 2D MR localizer images comprises:
removing outliers from the detected most likely positions of the liver dome and from the detected most likely positions of the right lobe lower tip; and
selecting an uppermost one of the most likely positions of the liver dome and a lowermost one of the most likely positions of the right lobe lower tip to define a search range that encompasses all of the other detected most likely positions of the liver dome and the right lobe lower tip.

11. The method of claim 1, further comprising:
receiving the plurality of 2D MR localizer images.

12. The method of claim 1, further comprising:
performing a diagnostic MR scan of the target organ using the determined scan range.

13. An apparatus for detecting a scan range for a magnetic resonance (MR) scan of a target organ based on a plurality of 2D MR localizer images, comprising:
means for detecting a most likely position of each of a plurality of anatomical landmarks associated with a target organ in each of the plurality of 2D MR localizer images; and
means for determining a scan range for the target based on the detected most likely positions of each of the plurality of anatomical landmarks in the plurality of 2D MR localizer images.

14. The apparatus of claim 13, wherein the means for detecting a most likely position of each of a plurality of anatomical landmarks associated with a target organ in each of the plurality of 2D MR localizer images comprises:
means for detecting one or more most likely position candidates for each anatomical landmark in each of the plurality of 2D MR localizer images using a separate trained landmark detector for each anatomic landmark.

15. The apparatus of claim 14, wherein the means for detecting a most likely position of each of a plurality of anatomical landmarks associated with a target organ in each of the plurality of 2D MR localizer images further comprises:
means for selecting one of the most likely position candidates for each anatomical landmark based on a relationship between the anatomical landmarks using a discriminative anatomical network.

16. The apparatus of claim 13, wherein the means for determining a scan range for the target based on the detected most likely positions of each of the plurality of anatomical landmarks in the plurality of 2D MR localizer images comprises:
means for removing outliers from the detected most likely positions for each of the plurality of anatomical landmarks; and
means for selecting one of the most likely positions for each of the anatomical landmarks to define a search range, such that the search range defined by the selected most likely position of each of the anatomical landmarks encompasses all of the other detected most likely positions for each anatomical landmark.

17. The apparatus of claim 13, wherein the target organ is a liver and the plurality of anatomical landmarks comprises a liver dome and a right lobe lower tip.

18. The apparatus of claim 17, wherein the means detecting a most likely position of each of a plurality of anatomical landmarks associated with a target organ in each of the plurality of 2D MR localizer images comprises:
means for detecting one or more most likely position candidates for the liver dome in each of the plurality of 2D MR localizer images using a trained liver dome detector;
means for detecting one or more most likely position candidates for the right lobe lower tip in each of the plurality of 2D MR localizer images using a trained right lobe lower tip detector constrained based on the detected most likely position candidates for the liver dome; and
means for selecting one of the most likely position candidates for the liver dome and one of the most likely position candidates for the right lobe lower tip using a discriminative anatomical network.

19. The apparatus of claim 17, wherein the means for determining a scan range for the target based on the detected most likely positions of each of the plurality of anatomical landmarks in the plurality of 2D MR localizer images comprises:
means for removing outliers from the detected most likely positions of the liver dome and from the detected most likely positions of the right lobe lower tip; and
means for selecting an uppermost one of the most likely positions of the liver dome and a lowermost one of the most likely positions of the right lobe lower tip to define a search range that encompasses all of the other detected most likely positions of the liver dome and the right lobe lower tip.

20. The apparatus of claim 13, further comprising:
means for receiving the plurality of 2D MR localizer images.

21. The apparatus of claim 13, further comprising:
means for performing a diagnostic MR scan of the target organ using the determined scan range.

22. A non-transitory computer readable medium encoded with computer executable instructions for detecting a scan range for a magnetic resonance (MR) scan of a target organ based on a plurality of 2D MR localizer images, the computer executable instructions defining steps comprising:
detecting a most likely position of each of a plurality of anatomical landmarks associated with a target organ in each of the plurality of 2D MR localizer images; and
determining a scan range for the target based on the detected most likely positions of each of the plurality of anatomical landmarks in the plurality of 2D MR localizer images.

23. The computer readable medium of claim 22, wherein the computer executable instructions defining the step of detecting a most likely position of each of a plurality of anatomical landmarks associated with a target organ in each of the plurality of 2D MR localizer images comprise computer executable instructions defining the step of:
detecting one or more most likely position candidates for each anatomical landmark in each of the plurality of 2D MR localizer images using a separate trained landmark detector for each anatomic landmark.

24. The computer readable medium of claim 22, wherein the computer executable instructions defining the step of detecting a most likely position of each of a plurality of anatomical landmarks associated with a target organ in each of the plurality of 2D MR localizer images further comprise computer executable instructions defining the step of:
selecting one of the most likely position candidates for each anatomical landmark based on a relationship between the anatomical landmarks using a discriminative anatomical network.

25. The computer readable medium of claim 2, wherein the computer executable instructions defining the step of determining a scan range for the target based on the detected most likely positions of each of the plurality of anatomical landmarks in the plurality of 2D MR localizer images comprise computer executable instructions defining the steps of:
removing outliers from the detected most likely positions for each of the plurality of anatomical landmarks; and
selecting one of the most likely positions for each of the anatomical landmarks to define a search range, such that the search range defined by the selected most likely position of each of the anatomical landmarks encompasses all of the other detected most likely positions for each anatomical landmark.

26. The computer readable medium of claim 22, wherein the target organ is a liver and the plurality of anatomical landmarks comprises a liver dome and a right lobe lower tip.

27. The computer readable medium of claim 26, wherein the computer executable instructions defining the step of detecting a most likely position of each of a plurality of anatomical landmarks associated with a target organ in each of the plurality of 2D MR localizer images comprise computer executable instructions defining the steps of:
detecting one or more most likely position candidates for the liver dome in each of the plurality of 2D MR localizer images using a trained liver dome detector;
detecting one or more most likely position candidates for the right lobe lower tip in each of the plurality of 2D MR localizer images using a trained right lobe lower tip detector constrained based on the detected most likely position candidates for the liver dome; and
selecting one of the most likely position candidates for the liver dome and one of the most likely position candidates for the right lobe lower tip using a discriminative anatomical network.

28. The computer readable medium of claim 26, wherein the computer executable instructions defining the step of determining a scan range for the target based on the detected most likely positions of each of the plurality of anatomical landmarks in the plurality of 2D MR localizer images comprise computer executable instructions defining the steps of:
removing outliers from the detected most likely positions of the liver dome and from the detected most likely positions of the right lobe lower tip; and
selecting an uppermost one of the most likely positions of the liver dome and a lowermost one of the most likely positions of the right lobe lower tip to define a search range that encompasses all of the other detected most likely positions of the liver dome and the right lobe lower tip.
